(19) United States
(12) Patent Application Publication
(10) Pub. No.: US 2016/0017003 A1
(43) Pub. Date: Jan. 21, 2016

(54) Process for the Production of Cyclosporin-A Using the Fungus Tolypocladium Sp. Strain NRRL No.: 18950

(71) Applicant: INDIAN COUNCIL OF MEDICAL RESEARCH, New Delhi (IN)

(72) Inventors: A. Mary Manonmani, Puducherry (IN); Irudayaraj Geetha, Puducherry (IN); Kothandapani Balaraman, Puducherry (IN)

(21) Appl. No.: 14/774,967

(22) PCT Filed: Mar. 12, 2014
(86) PCT No.: PCT/IN2014/000160

§ 371 Date: Sep. 11, 2015

(30) Foreign Application Priority Data
Mar. 12, 2013 (IN) 699/DEL/2013

(51) Int. Cl. C07K 7/64; C12R 1/645

(52) U.S. Cl. CPC C07K 7/645; C12R 1/645

(57) ABSTRACT

Provided herein is a process for the production of Cyclosporin-A (Cyc-A) including the steps of inoculating a nutrient medium with the fungus Tolypocladium sp., strain NRRL No. 18950 followed by cultivation under static conditions to obtain a fermented medium with the fungal biomass, and harvesting the biomass and subjecting the harvested biomass to extraction followed by purification to obtain pure Cyc-A. The nutrient medium includes glucose, glycerol, casein acid hydro lysate, malt extract, peptone, and L-valine.

### FIELD OF THE INVENTION

This invention relates to a process for the production of Cyclosporin-A (Cyc-A), an immunosuppressive agent from Tolypocladium sp. (NRRL No: 18950). This immunosuppressive agent has the potential mainly to prevent organ rejection in transplantation surgery.

### BACKGROUND OF THE INVENTION

Cyclosporins have been reported to be synthesized by the fungus, Tolypocladium terricola through stationary cultivation (Matha et al., 1993). The Vector Control Research Centre has reported the production of Cyclosporin-A (Cyc-A) by cultivating the fungus, Tolypocladium sp. (NRRL No.18950) under static conditions and the mean yield was found to range from 582 mg to 1256 mg per litre of culture medium [European Patent EP-96-356060/36 (1996), U.S. Pat. No. 5,656,459 (1997), Brazilian Patent BR9601017 (1997), Indian Patents 183940 (2000), German Patent DE69521193T (2001), Canadian Patent CA2142240C (2002); Balaraman & Mathew, 2006]. There are also several reports on synthesis of Cyc-A through submerged and solid state cultivation of Tolypocladium sp. (Agathos et al., 1986; Lee & Agathos, 1989; Agathos & Perekh, 1990; Issac et al., 1990; Aarnio & Agathos, 1990; Lee & Agathos, 1991; Ramana murthy et al., 1993 & 1999; Sekar et al., 1997; Sekar & Balaraman, 1998a; Survase et al., 2009a & b). However, the need exists, to modify the processes whereby the up-stream and down-stream processing is changed, to improve the process and provide greater yields.

### OBJECTS OF THE INVENTION

It is therefore an object of this invention to propose a process for the production of Cyclosporin-A, which is cost-effective.

It is a further object of this invention to propose a process for the production of Cyclosporin-A, which is simple and uses easily available materials.

These and other objects and advantages of the invention will be apparent to a person on reading the ensuing description, when read in conjunction with the accompanying drawings:

### BRIEF DESCRIPTION OF THE ACCOMPANYING DRAWINGS

FIG. 1: Flow sheet depicting the process for the preparation of Cyclosporin-A by static cultivation.

### DETAILED DESCRIPTION OF THE INVENTION:

According to this invention is provided a process for the production of Cyclosporin-A (cyc-A) using the fungus Tolypocladium sp., NRRL No. 18950.

In accordance with this invention there is provided a process for the production of Cyclosporin-A (Cyc-A) comprising the steps of inoculating a nutrient medium with the fungus Tolypocladium sp., strain NRRL No. 18950 followed by cultivation under static conditions to obtain a fermented medium with the fungal biomass, harvesting the biomass and subjecting the harvested biomass to treatment with hot water followed by freezing the biomass to release the intracellularly synthesized Cyc-A, subjecting the biomass to extraction followed by purification to obtain pure Cyc-A, wherein said nutrient medium comprises glucose, glycerol, casein acid hydrolysate, malt extract, peptone, L-valine.

In accordance with this invention, the production strain of the fungus was revived from the culture collection of Vector Control Research Centre (VCRC). For the inoculation of production medium, inoculum (seed) was prepared in two stages: Mycelial growth from the slant was transferred to 50 ml of a first seed production medium containing glucose 2-5%, peptone 0.5-2.5%, casein acid hydrolysate 0.5-2% (pH 5-6) and incubated on shaker at 50-200 rpm for about 2-4 days at 22-24° C. to obtain the first stage seed. Second stage seed is prepared by transferring the first stage seed to 200 ml of a second seed production medium mentioned above and incubated on shaker at 50-200 rpm for about 2-5 days at 22-24° C. The second stage seed or the inoculum was inoculated to the Cyc-A production medium (PM) (Composition: 2.0-4.5% of glucose, 2.0-4.5% of glycerol, 1.0-3.0% of casein acid hydrolysate, 0.5-2.0% of malt extract, 0.2-1.0% of peptone, 0.1-0.5% of L-valine per liter of distilled water at 5-20% level and incubated under static conditions for 15-25 days at 22-24° C. The proportions mentioned herein are all in percentages by weight.

After the incubation period, the biomass of the Tolypocladium fungus is filtered through country filter paper and subjected to treatment with hot water (at 100-110° C.) for 5-15 min so as to inactivate the enzymes involved in the oxidation of phenolic compounds and dried over filter paper for 10-15 min. Then, the biomass is frozen at −50 to −80° C. for 18-24 h so as to release the intracelluarly synthesized Cyc-A. The frozen biomass is thawed at room temperature and subjected to blending with the extractant, methanol and incubated on shaker overnight at 250 rpm. The homogenate is then vacuum-filtered through Whatman no. 1 filter paper and the filtrate is evaporated, residue reconstituted in sufficient quantity of distilled water, which is subjected to liquid-liquid extraction using ethyl acetate. The ethyl acetate fraction thus obtained is washed sequentially with sodium bicarbonate (2-5%) and distilled water to remove pigments and the decolourised ethyl acetate fraction is evaporated to obtain a residue.

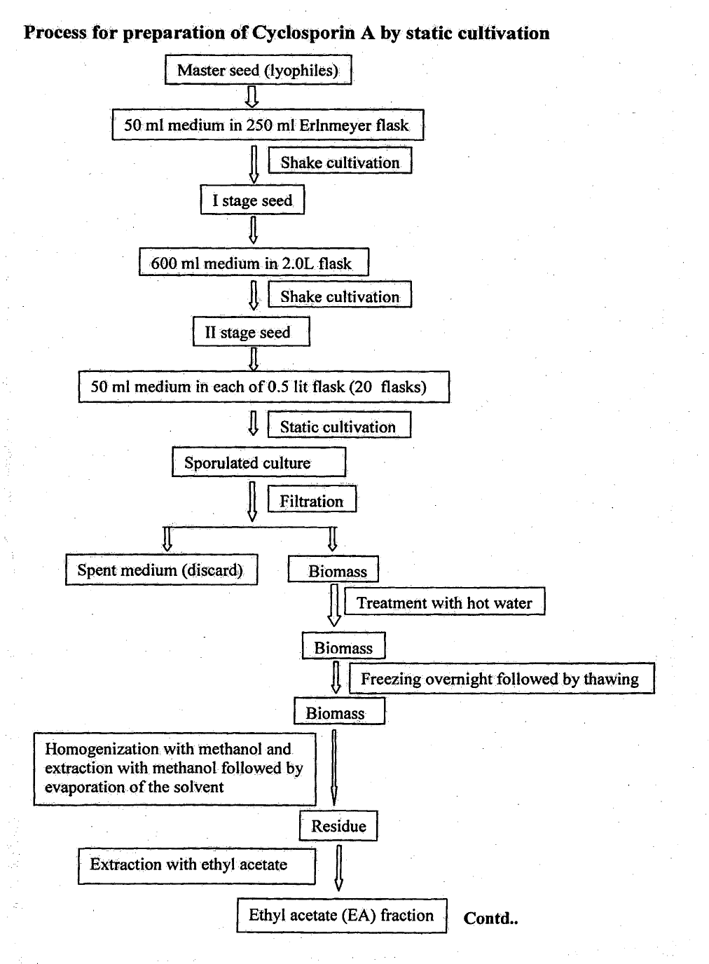

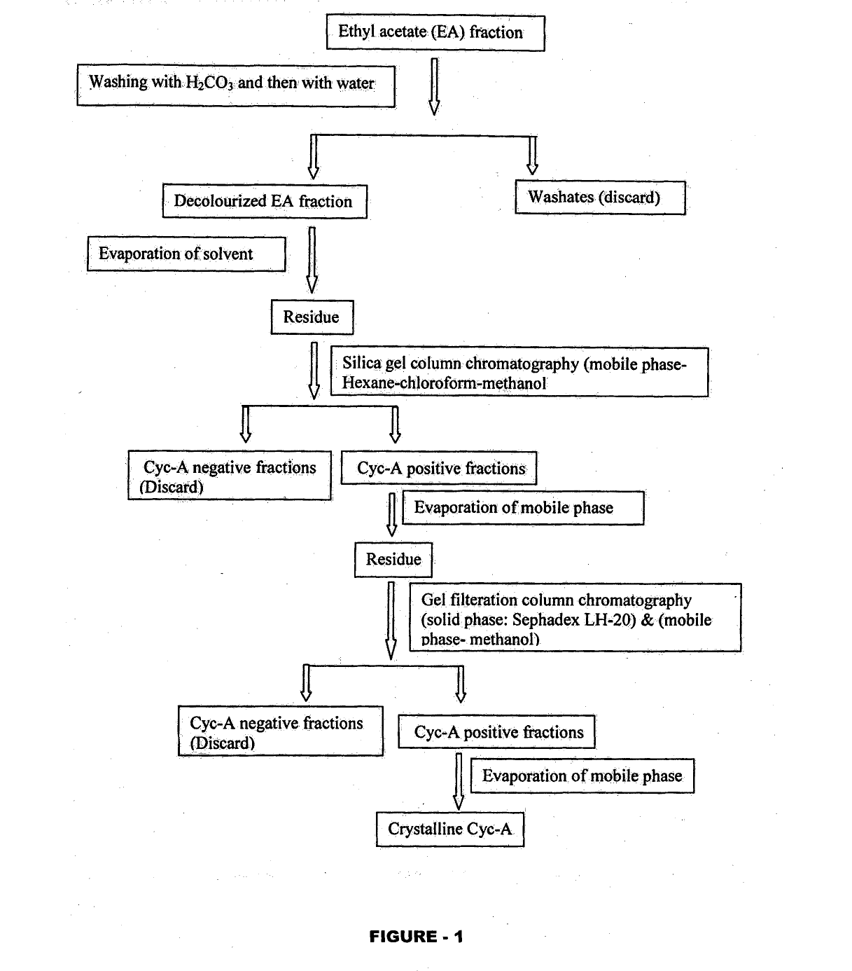

The residue is dissolved in a mixture of hexane, chloroform and methanol (at the ratio of 10:9:1) and subjected to column chromatography using silica gel as solid phase and a mixture of hexane, chloroform and methanol (at the ratio of 10:9:1) as mobile phase. The Cyc-A positive fractions, as determined by HPLC (George et al., 1992) are pooled and the solvent is evaporated to obtain a residue. This residue is dissolved in methanol and subjected to a step of column chromatography using the resin, LH-20 as solid phase and methanol as mobile phase. The Cyc-A positive fractions (as determined by HPLC) are pooled, the mobile phase is evaporated to obtain Cyc-A crystals whose purity is determined through HPLC (Flow chart—FIG. 1). The invention will now be explained in greater details with the help of the following non-limiting examples:

### EXAMPLE 1

First Stage Inoculum Development:

The fungus Tolypocladium sp., strain NRRL No. 18950, maintained in the culture collection of VCRC was revived and grown on nutrient agar slants containing
Glucose 2%
Peptone 1%
Casein acid hydrolysate 2% (pH 5-6)
Agar 2%

Fungal growth from slant was scrapped and inoculated to 50 ml of nutrient medium. The inoculated nutrient medium was incubated on a rotary shaker at 150 rpm for 4 days at 25° C. The 4 day old culture (I stage seed) was used to inoculate II stage nutrient medium.

### EXAMPLE 2

Second Stage Inoculum Development for the Production of Cyclosporin A:

First stage seed of fungal strain Tolypocladium sp. (NRRL NO. 18950) obtained from Example 1 was inoculated to 200 ml of nutrient medium with the same composition as that of Seed I at 25% level. The inoculated nutrient medium was incubated on a rotary shaker at 110 rpm for 3 days at 25 ° C. The 3 day old culture (I stage seed) was used as inoculum for the production medium.

### EXAMPLE 3

Production of Cyclosporin A by Static Cultivation:

Second stage seed developed as given in Example 2 was inoculated to the production medium containing the following ingredients at 5% level and incubated at 25° C. under static condition for 21 days.
Glucose—4%
Glycerol—4%
Casein acid hydrolysate—3%
Malt extract—2%
Peptone—1%
L-valine—0.5%

### EXAMPLE 4

Seperation of Fungal Biomass:

The biomass of the fungal strain Tolypocladium sp. (NRRL NO. 18950) used for the extraction of Cyclopsorin A was harvested from the production medium given in Example 3. On 21^{st} day of incubation the culture was taken from the incubator and the biomass separated by filteration. The separated biomass was subjected to treatment with hot water (at 100-110° C.) for 5 min. The treated biomass was frozen at −80° C. for 24 h and then thawed at room temperature.

### EXAMPLE 5

Extraction and Separation of Cyclosporin A from the Fungal Biomass:

The biomass of the fungal strain Tolypocladium sp. (NRRL NO. 18950) mentioned in Example 4 was subjected to blending with the extractant, methanol and incubated on shaker overnight at 250 rpm at room temperature. The homogenate was then vacuum-filtered through Whatman no. 1 filter paper and the filtrate was evaporated. The methanol residue obtained was reconstituted in sufficient quantity of distilled water, which was subjected to liquid-liquid extraction using ethyl acetate. The ethyl acetate fraction thus obtained was washed sequentially with sodium bicarbonate (5%) and distilled water to remove pigments and the decolourised ethyl acetate fraction was evaporated to obtain a cyclosporin containing residue.

### EXAMPLE 6

Purification of Cyclosporin A by Column Chromatography:

The ethyl acetate residue obtained in Example 5 was dissolved in a mixture of hexane, chloroform and methanol (at the ratio of 10:9:1) and subjected to column chromatography using silica gel as solid phase and a mixture of hexane, chloroform and methanol (at the ratio of 10:9:1) as mobile phase. The Cyc-A positive fractions, as determined by HPLC (George et al., 1992) were pooled and the solvent was evaporated to obtain a residue. This residue was dissolved in methanol and subjected to a step of column chromatography using the resin, LH-20 as solid phase and methanol as mobile phase. The Cyc-A positive fractions (as determined by HPLC) were pooled, the mobile phase was evaporated to obtain Cyc-A crystals whose purity was determined through HPLC.

### EXAMPLE 7

Use of DL-amino Butyric Acid (ABU) as Precursor:

In one set of experiments, the fungal strain Tolypocladium sp. (NRRL No. 18950) was cultured by static cultivation method using DL-amino butyric acid (ABU) as precursor. After the incubation period of 21 days the fungal biomass was extracted using solvents, purified through column chromatography and Cyc A crystals were obtained. Purity of Cyc A was analysed through HPLC. The yield and purity of Cyc A obtained from these experiments are presented in Table 1.

### EXAMPLE 8

Use of L-valine as Precursor:

In another set of experiments the fungal strain Tolypocladium sp. (NRRL NO. 18950) was cultured by static cultivation method using L-Valine as precursor. After the incubation period of 21 days, the fungal biomass was extracted with the modified extraction procedure wherein, the fungal biomass was subjected to hot water treatment followed by freezing. The frozen biomass was extracted with solvents using the previously reported protocol, purified through column chromatography and Cyc A crystals were obtained. Purity of Cyc A was analysed through HPLC. The yield of Cyc-A per litre of the culture medium and the purity of Cyc-A obtained are presented in Table 1.

TABLE 1
Yield and Purity of Cyc-A using ABU/or L-valine
as precursor under static cultivation

| | | | | Specific | | | |
|---|---|---|---|---|---|---|---|
| | | | | production of | | | |
| | | | | Cyc A (mg of | | | |
| | | Cyc-A yield | | Cyc-A/g of | | | |
| Batch | Batch | (mg) | | biomass) | | Purity (%) | |
| No. | size | ABU | L-valine | ABU | L-valine | ABU | L-valine |
| 1 | 1 lt | 601 | 1189 | 3.41 | 4.91 | 90.6 | 90.9 |
| 2 | 1 lit | 720 | 1237 | 3.10 | 5.22 | 90.6 | 91 |
| 3 | 1 lit | 808 | 1031 | 3.16 | 3.64 | 90.9 | 91 |
| 4 | 1 lit | 800 | 1066 | 3.10 | 3.54 | 90.6 | 91 |
| 5 | 1 lit | 1059 | 850 | 4.30 | 3.23 | 88.4 | 91.2 |

| | | | | | | |
|---|---|---|---|---|---|---|
| Average | 797.6 | 1074.6 | 3.41 | 4.11 | 90.1 | 91.0 |

Increase in Cyc-A yield using L-valine as precursor-34.7%

According to this invention, a new precursor, L-valine was used in place of ABU for directing the synthesis of Cyc-A in the production medium using the new precursor. It was observed that the weight of fungus biomass as well as the yield of purified Cyc-A was higher when L-valine was used in place of ABU; the specific production of Cyc A (mg of Cyc-A/g of biomass) was higher when L-valine was used compared to ABU. Also, the purity of Cyc A was slightly higher when L-valine was used compared to ABU and the cost of Cyc-A production was brought down by 4-5 times while using L-valine instead of ABU.

1. A process for the production of Cyclosporin-A (Cyc-A) comprising the steps of: inoculating a nutrient medium with the fungus Tolypocladium sp., strain NRRL No. 18950, followed by cultivation under static conditions to obtain a fermented medium with a fungal biomass; and harvesting the biomass and subjecting the harvested biomass to extraction followed by purification to obtain pure Cyc-A, wherein said nutrient medium comprises glucose, glycerol, casein acid hydrolysate, malt extract, peptone, and L-valine.

2. The process as claimed in claim 1, wherein the concentration of the precursor L-valine in the nutrient medium is 0.5%.

3. The process as claimed in claim 1, wherein the harvested biomass is subjected to treatment with hot water followed by freezing to release the intracellularly synthesized Cyc-A.

4. The process as claimed in claim 3, wherein said hot water treatment is effected at a temperature range of 100-110° C.

5. The process as claimed in claim 3, wherein said hot water treatment is effected for 5 minutes.

6. The process as claimed in claim 3, wherein the step of freezing the biomass is effected at a temperature range of −50 to −80° C.

7. The process as claimed in claim 3, wherein the step of freezing the biomass is effected for 24 hours.

8. The process as claimed in claim 1, wherein the yield of Cyc-A is increased by 34.7% by addition of L-valine as compared to yield of Cyc-A when DLamino butyric acid is used.
